(19) United States
(12) Patent Application Publication
(10) Pub. No.: US 2003/0013883 A1
(43) Pub. Date: Jan. 16, 2003

(54) Tropane analogs binding to monoamine transporters

(76) Inventors: Gilles D. Tamagnan, Woodbridge, CT (US); Xing Fu, West Haven, CT (US); Ronald Martin Baldwin, Guilford, CT (US); Robert Innis, Rockville, MD (US)

Correspondence Address: Docket Coordinator, WIGGIN & DANA, One Century Tower, 265 Church Street, New Haven, CT, 06508-1832, US

(21) Appl. No.: 09/878,450

(22) Filed: Jun. 11, 2001

Related U.S. Application Data
(60) Provisional application No. 60/211,989, filed on Jun. 16, 2000.

(51) Int. Cl. C07D 451/02

(52) U.S. Cl. 546/124; 546/125

(57) ABSTRACT

The present invention is directed to a tropane analog having the structure (I)

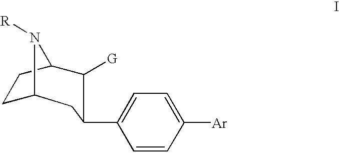

wherein Ar is a selected substituted or unsubstituted aryl group; G═CO₂CH₃, COR, or CH═CHR; X═H, I, Br, Cl, F, NO₂, COR, or, NH₂; R═H or a substituted or unsubstituted lower alkyl group; and Z═O, S, or N. The tropane analogs of the invention bind to monoamine transporters in the mammalian brain, and are useful as diagnostic tools for analyzing the role of monoamine transporters in diseases such as depression, schizophrenia, major depression, attention-deficit hyperactivity disorder, obesity, obsessive compulsive disorders, and cocaine addiction.

This application claims the benefit of Provisional Patent application No. 60/211,989 filed Jun. 16, 2000.

### STATEMENT OF GOVERNMENT SUPPORT

This invention was made in part with government support under grant number P30-MH30929 from the National Institutes of Mental Health. The government has certain rights in this invention.

### BACKGROUND OF THE INVENTION

1. Field of the Invention

The present invention is generally directed to tropane analogs, and more particularly to tropane analogs having the structure (I)

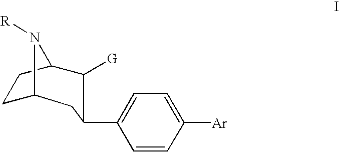

wherein Ar is a selected substituted or unsubstituted aryl group or a selected substituted or unsubstituted heterocyclic group; G is CO₂R, COR, CH₂OR, or CH═CHR; X is H, I, Br, Cl, F, NO₂, COR, or, NH₂; R and R′ are H or a substituted or unsubstituted lower alkyl group; and Z═O, S, or N.

2. Description of the Related Art

The brain consists of a plurality of neurons that interact by exchanging chemical messengers. Each neuron generates neurochemicals, referred to as neurotransmitters; neurotransmitters act at sites on the cellular membrane of a neuron, the sites being referred to as receptors. Receptors are associated with either ion channels through the cellular membrane or secondary neurochemical messenger systems. By contrast, transporters (also known as reuptake sites) are molecular complexes which transport chemicals across the cellular membrane of a neuron. When a neurotransmitter has served its function, it is removed from the vicinity of the receptor by being bound to a transporter which transports the neurotransmitter to the interior of the neuron.

Just as there are many specialized neurons in the brain, there are also a variety of neurotransmitters, associated receptors, and transporters. The distribution of specialized neurons depends upon the particular organism under study, and the state of health of that organism. A neuron can be classified according to the type of neurotransmitter that it uses to communicate with other neurons. Certain types of neurons can be found predominantly in particular regions of the brain. For example, the striatal region of a mammalian brain is innervated by neurons using dopamine as a neurotransmitter. Certain compounds, such as cocaine, have a preferential affinity for dopamine transporters, and therefore tend to bind to such transporters. The effect of a molecule such as cocaine upon a dopamine transporter is to inhibit reuptake of the neurotransmitter dopamine, leaving more dopamine available in the vicinity of the dopamine receptors.

In certain neurological diseases, such as Parkinson's disease, distinct groups of neurons lose their normal physiological functioning. Consequently, the abnormal neurons may behave differently in the presence of some neurotransmitters, and may also produce neurotransmitters in a manner that differs from a healthy neuron. The major neurotransmitters, dopamine, norepinephrine, and serotonin, are referred to collectively as the monoamine neurotransmitters. Many neurons have receptors and transporters adapted to receive at least one of these neurotransmitters. Parkinson's disease is caused by the degeneration of some of the dopaminergic neurons in the brain. The neurons lost in Parkinson's disease have a large number of dopamine transporters; cocaine and chemical analogs of cocaine have an affinity for such transporters.

A radioisotope is commonly incorporated in molecules that have a demonstrated binding affinity for a particular type of neuroreceptor, and such molecules are commonly used as neuroprobes. The localization of neuroprobes can be used to find specialized neurons within particular regions of the brain. It is also known that a neurological disease can be detected by observing abnormal binding distributions of a neuroprobe. Such abnormal binding distributions can be observed by incorporating a radionuclide within each molecule of the neuroprobe with a high binding affinity for the particular reuptake site or transporter of interest. Following binding, an imaging technique can be used to obtain a representation of the in vivo spatial distribution of the reuptake sites of interest.

In single photon emission computed tomography (SPECT) imaging, the most commonly used radionuclides are heavy metals, such as ^{99m}Tc. However, heavy metals are very difficult to incorporate into the molecular structure of neuroprobes because such probes are relatively small molecules (molecular weight less than 400). In positron emission tomography (PET), the radiohalide ¹⁸F (fluorine) is commonly used as a substitute for H (hydrogen) in radiopharmaceuticals because it is similar in size. Not all halogens will work, however. For example, I (iodine) is much larger than both H and F, being approximately half the size of a benzene ring. However, due to the small size of typical radiopharmaceuticals for use as neuroprobes, the presence of iodine markedly changes the size of the compound, thereby altering or destroying its biological activity. In addition, the presence of iodine in a neuroprobe tends to increase its lipophilicity, and therefore increases the tendency of the neuroprobe to engage in non-specific binding. For example, paroxetine is a drug with high affinity and selectivity for serotonin reuptake sites, and [³H]paroxetine has been shown in rodents to be a useful in vivo label (Scheffel, U. and Hartig, P. R. J. Neurochem. 52:1605-1612, 1989). However, several iodinated analogs of this compound with iodine attached at several different positions had unacceptably low affinity, in fact being one tenth of the affinity of the parent compound. Furthermore, when the iodinated compound was used as an in vivo radiolabeled neuroprobe, non-specific binding activity was found to be so high that no measurable portion of the brain uptake appeared to be specifically bound to the serotonin reuptake site. Thus, the iodinated form of paroxetine is not useful as an in vivo probe.

An iodinated compound can be useful as an in vitro probe, but may be useless as an in vivo probe, because an in vivo probe must meet the requirements associated with intravenous administration of the probe to a living subject. Reasons for the loss of in vivo utility include the fact that the compound may be metabolized too quickly, that it may not cross the blood-brain-barrier, and that it may have high non-specific uptake into the lipid stores of the brain. In vitro homogenate binding studies remove these obstacles by isolating the brain tissue from hepatic metabolic enzymes, by homogenizing the brain tissue so as to destroy the blood-brain-barrier, and by diluting the brain tissue so as to decrease the concentration of lipids in the assay tube. Accordingly, it cannot be assumed that a probe will be useful in both in vivo and in vitro modalities.

The tropane skeleton is a basic structural unit that can lead to compounds with diverse central nervous system activity. Due to the rigid nature of the structure, the possibility exists for the preparation of highly selective compounds. Representative patents pertaining to tropane derivatives include U.S. Pat. Nos. 5,268,480; 5,496,953; 5,506,359; 5,760,055; 5,750,089; 5,700,446; 5,698,179; 5,439,666 and 5,310,912. However, as is evident from the above discussion, there is a need in the art for improved tropane analogs for use as research tools, and as potential compounds for treatment of diseases. This invention is believed to be an answer to that need.

### SUMMARY OF THE INVENTION

In one aspect, the present invention is directed to a tropane analog having the structure (I)

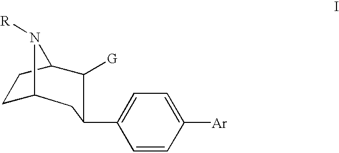

wherein Ar is linked to structure (I) through the dotted line and is selected from the group consisting of

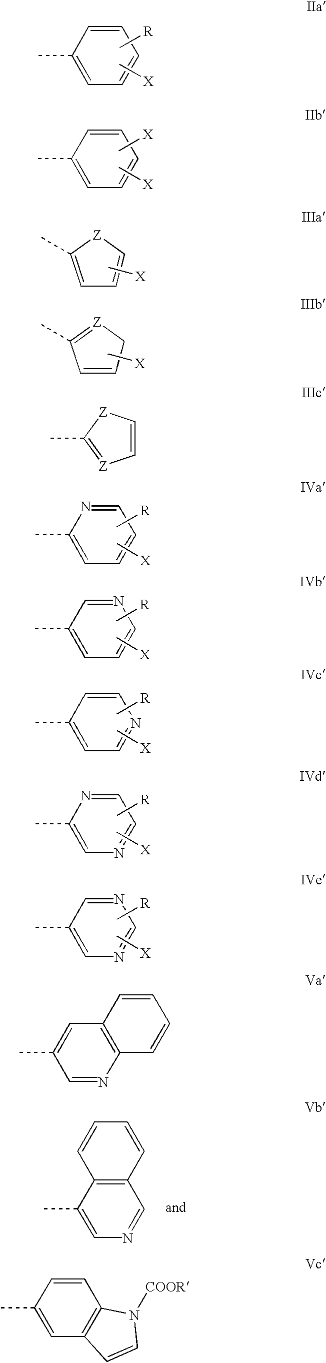

wherein Ar is a selected substituted or unsubstituted aryl group or a selected substituted or unsubstituted heterocyclic group; G is CO₂R, COR, CH₂OR, or CH═CHR; X is H, I, Br, Cl, F, NO₂, COR, or, NH₂; R and R′ are each individually H or a substituted or unsubstituted lower alkyl group; and Z═O, S, or N.

The Ar group in structure (I) above may be a substituted or unsubstituted phenyl group, a heterocyclic 5-ring structure, a heterocyclic 6-ring structure, or a heterocyclic 10-ring structure. The present invention is also directed to use of the above tropane analogs in assays of the monoamine transporters in the mammalian brain.

These and other aspects will be described in more detail in the following detailed description of the invention.

### DETAILED DESCRIPTION OF THE INVENTION

It has now been surprisingly found that aromatic substituted phenyltropane derivatives having the following general structure

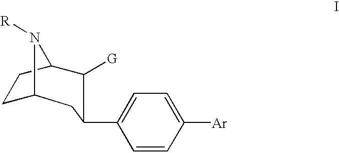

wherein Ar is a selected substituted or unsubstituted aryl group or a selected substituted or unsubstituted heterocyclic group; G is CO₂R, COR, CH₂OR, or CH═CHR; X is H, I, Br, Cl, F, NO₂, COR, or, NH₂; R and R′ are each individually H or a substituted or unsubstituted lower alkyl group; and Z═O, S, or N exhibit binding affinity towards monoamine transporters. The aromatic substituted phenyltropane derivatives of the present invention have been found to selectively bind to monoamine transporters and thus have the potential for the treatment of major depression, attention-deficit hyperactivity disorder, obesity, obsessive compulsive disorders, and cocaine addiction. The aromatic substituted phenyltropane derivatives of the present invention are also useful as diagnostic tools for analyzing the role of monoamine transporters in diseases such as depression and schizophrenia.

As indicated above, the aromatic substituted phenyltropane derivatives having the following general structure (I):

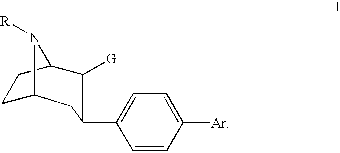

In the above general structure, Ar is a selected substituted or unsubstituted aryl group or a substituted or unsubstituted heterocyclic group; G is CO₂R, COR, CH₂OR or CH═CHR; X is H, I, Br, Cl, F, NO₂, COR, or, NH₂; R and R′ are each individually H or a substituted or unsubstituted lower alkyl group; and Z═O, S, or N. The Ar moiety is selected from the group consisting of structures IIa′ or b′, IIIa′, b′, or c′, IVa′, b′, c′, d′, or e′, or Va′, b′, or c′ as follows:

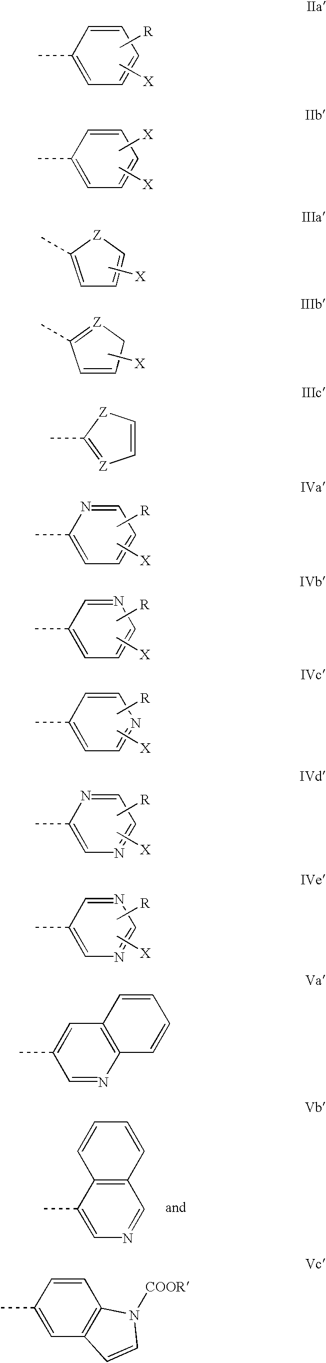

As used herein, the term “lower alkyl group” refers to straight-chain, branched, or cyclo alkyl groups containing 1 to 6 carbon atoms. Examples of such groups include, but are not limited to, methyl, ethyl, propyl, pentyl, hexyl, isopropyl, sec butyl, tert butyl, isopentyl, cyclobutyl, cyclopropyl, and the like. The term “substituted” refers to a hydrogen atom being replaced with another functional group. Examples of such substituted functional groups include hydroxy, carboxy, methyl, ethyl, nitrile, and the like. In addition, the halogen or other atoms of group X may be any isotope, including heavy or radioactive isotopes. Useful examples of such isotopes include ³H (tritium), ¹²³I, ¹²⁵I, ¹³¹I, ¹⁸F, ⁷⁶Br, ¹¹C, ¹⁴C, and the like.

Depending on the choice of the Ar moiety in the general structure (I) above, groups of substructures may be formed, in particular biphenyl structures, heterocyclic 5-ring structures, heterocyclic 6-ring structures, or heterocyclic 10-ring structures. Each of these structures is discussed in more detail below.

Biphenyl structures have the following general structure (II):

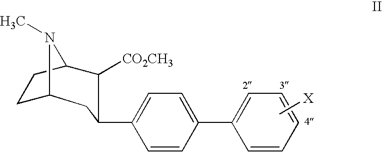

Examples of useful compounds having the general structure (II) above are shown in Table 1.

TABLE 1

| | General | | |
|---|---|---|---|
| Compound | Structure | Position | X |
| 1 | II | — | H |
| 2 | II | 3″ | OH |
| 3a | II | 2″ | OCH₃ |
| 3b | II | 3″ | OCH₃ |
| 3c | II | 4″ | OCH₃ |
| 4a | II | 2″ | NO₂ |
| 4b | II | 3″ | NO₂ |
| 4c | II | 4″ | NO₂ |
| 5 | II | 3″ | NH₂ |
| 6a | II | 2″ | COCH₃ |
| 6b | II | 3″ | COCH₃ |
| 6c | II | 4″ | COCH₃ |

Additional useful biphenyl structures include structures 7-10 below:

TABLE 2

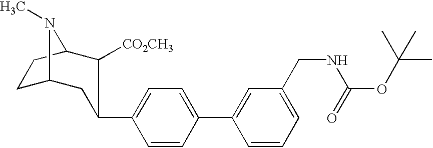

7

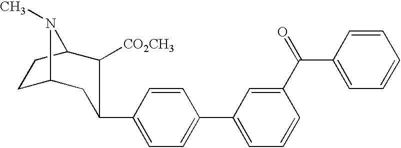

8

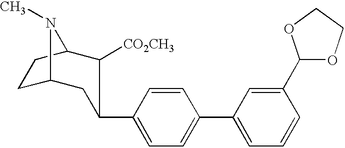

9

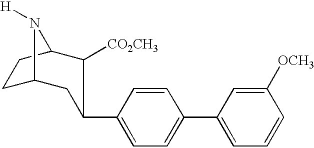

10
Structures 7-10 are summarized in Table 2:

| Compound | Position | X |
|---|---|---|
| 7 | 3″ | CH₂NH-tBoc |
| 8 | 3″ | COPh |
| 9 | 3″ | CH(OCH₂)₂ |
| 10 | 3″ | OCH₃ |

Heterocyclic 5-ring structures have the following general structures IIIa, IIIb, and IIIc:

TABLE 3

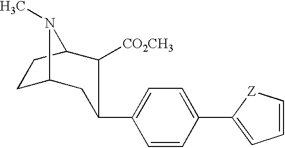

IIIa

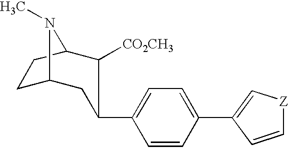

IIIb

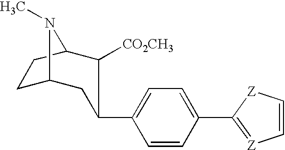

IIIc
Examples of useful compounds having the general structures IIIa-c
above are shown in Table 3.

| | General | | |
|---|---|---|---|
| Compound | Structure | Position | Z |
| 11 | IIIa | 2″ | S |
| 12 | IIIb | 3″ | S |
| 13 | IIIa | 2″ | O |
| 14 | IIIc | 2″, 5″ | S, N |

Heterocyclic 6-ring structures have the following general structures IVa, IVb, and IVc:

TABLE 4

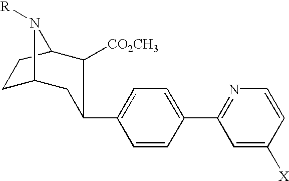

IVa

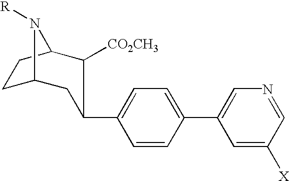

IVb

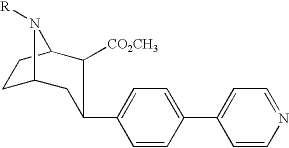

IVc
Examples of useful compounds having the general structures above
are shown in Table 4.

| | General | | | |
|---|---|---|---|---|
| Compound | Structure | R | Position | X |
| 15 | IVa | CH₃ | 2″ | H |
| 16 | IVb | CH₃ | 3″ | H |
| 17 | IVb | CH₃ | 4″ | OCH₃ |
| 18 | IVb | H | 3″ | OCH₃ |

Additional useful heterocyclic 6-ring structures include structure 19 below:

TABLE 5

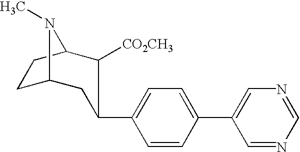

19
Examples of useful heterocyclic 10-ring structure compounds are shown
in Table 5.

| Compound | Type |
|---|---|
| 20 | Quinoline |
| 21 | Isoquinoline |
| 22 | Indole |

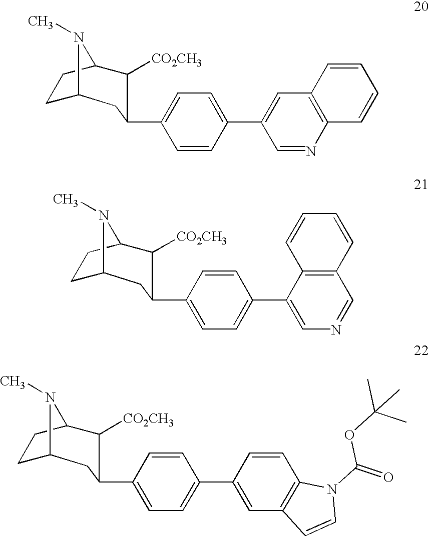

The tropane analogs of the present invention may be synthesized using conventional organic chemistry techniques known in the art, and the products may be analyzed using conventional methods, (NMR, IR, UV, mass spectroscopy, and the like). Examples of organic syntheses are explained in detail below.

### EXAMPLES

The following Examples are intended to illustrate, but in no way limit the scope of the present invention. All parts and percentages are by weight and all temperatures are in degrees Celsius unless explicitly stated otherwise.

### Example 1.

### Synthesis of (3β-Biphenyl-4-yl) Tropane-2β-Carboxylic Acid Methyl Ester (1)

Synthesis of this compound followed generally Scheme I, where R═CH₃, Z═C, and X═H.

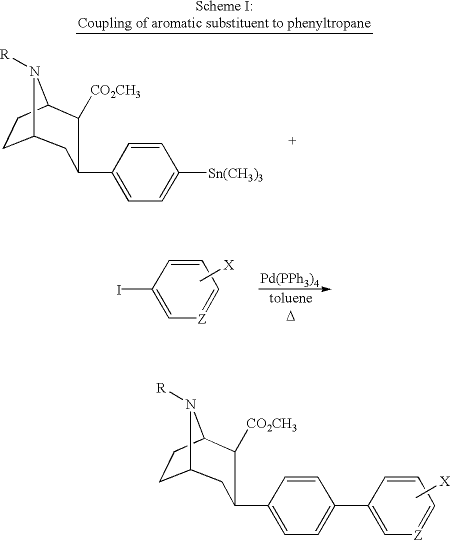

To a solution of 200 mg (0.475 mmol) of 3β-(4-trimethylstannylphenyl)tropane-2β-carboxylic acid methyl ester in 5 mL of dry toluene under a nitrogen atmosphere was added 116 mg (0.57 mmol) of iodobenzene followed by 32 mg tetrakis(triphenylphosphine)palladium, as catalyst. The mixture was heated at reflux for 18 h. The reaction mixture was purified by flash chromatography on silica gel. Elution with 50:50 hexane-ether with 5% additional triethylamine afforded 73 mg (48% yield) of (3β-biphenyl-4-yl) tropane-2β-carboxylic acid methyl ester. The product was analyzed as follows: ¹H NMR (CD₂Cl₂) δ 7.40 (d, 2H aryl H), 7.33 (d, 2H aryl H), 7.22 (t, 2H, aryl H), 7.13 (d,3H, aryl H), 3.37 (t, 1H), 3.28 (s,3H), 2.87-2.76 (m, 2H), 2.35 (dt, 1H), 2.06-1.90 (m, 5H), 1.58-1.39 (m, 3H). ¹³C NMR (CD₂Cl₂) δ 25.9, 26.5, 34.0, 34.7, 42.4, 51.6, 53.3, 63.2, 66.2, 127.1, 127.6, 127.8, 128.5, 129.5, 139.1, 141.7, 143.7, 172.7.

### Example 2.

### Synthesis of 3β-[4-(5′-Methoxypyridin-3-yl)-phenyl]nortropane-2β-Carboxylic Acid Methyl Ester (17).

Synthesis of this compound followed generally Scheme I above, where R═H, Z═N, X═OCH₃. To a solution of 120 mg (0.227 mmol) of 3β-[4-(5-methoxypyridin-3-yl)-phenyl]tropane-2β, 8-dicarboxylic acid 2-methyl ester 8-(2,2,2-trichloro-ethyl) ester in 3 mL of dry THF under a nitrogen atmosphere was added 3 mL of 1 M aqueous ammonium acetate followed by 152 mg (1.14 mmol) 10% Cd/Pb. The mixture was stirred at room temperature for 2 h and then filtered through a plug of Celite. The solution was basified with ammonium hydroxide and extracted with ether (5×). The combined extract was dried over magnesium sulfate, filtered, and concentrated under reduced pressure. The crude oil was purified by flash chromatography on silica gel. Elution with 85:15 ether-triethylamine afforded 50 mg (63% yield) of 3β-[4-(5′-methoxypyridin-3-yl)-phenyl]nortropane-2β-carboxylic acid methyl ester.

The product was analyzed as follows: ¹H NMR (CD₂Cl₂) δ 8.33 (d, 1H J=2 Hz), 8.15 (d, 1H J=2.8 Hz), 7.44 (d, 2H, J=8 Hz), 7.28 (q, 1H, J1=2 Hz, J2=2.6 Hz), 7.23 (d, 2H, J=8 Hz), 3.80 (s, 3H), 3.60 (d, 2H), 3.22 (s, 3H), 3.19 (m, 1H), 2.71 (m, 1H), 2.32 (dt, 1H), 1.97-1.83 (m, 2H), 1.72-1.1.54 (m, 3H).

### Example 3.

### Synthesis of 3β-[4-(5′-Methoxy-pyridin-3-yl)-phenyl]-tropane-2β-carboxylic acid methyl ester (16)

Synthesis of this compound followed generally Scheme I above, where R═CH₃, Z═N, X═OCH₃.

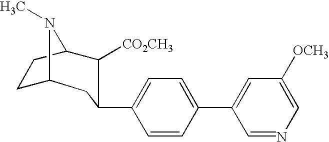

To a solution of 200 mg (0.475 mmol) of 3-(4-trimethylstannanyl-phenyl)-tropane-2β-carboxylic acid methyl ester in 5 mL of dry toluene under a nitrogen atmosphere was added 116 mg (0.57 mmol) of iodo benzene followed by 32 mg tetrakis (triphenylphosphine) palladium.

The mixture was heated to reflux for 18 h. The reaction mixture was purified by flash chromatography on silica gel. Elution with 20:80 hexane-ether with 5% additional triethylamine afforded 147 mg (85% yield) of 3β-[4-(5′-methoxy-pyridin-3-yl)-phenyl]-tropane-2β-carboxylic acid methyl ester. The compound was anayzed as follows: ¹H NMR (CD₂Cl₂) δ 8.40 (d, 1H), 8.22 (d, 1H, J=2.8 Hz), 7.50 (d, 2H, J=8 Hz), 7.36 (dd, 1H, J=2.8 Hz, J=1.6 Hz), 7.34 (d, 2H, J=8.4 Hz), 3.88 (s, 3H), 3.55 (q, 1H), 3.45 (s, 3H), 3.32 (m, 1H), 3.03 (m, 1H), 2.95 (m, 1H), 2.51 (dt, 1H), 2.21-2.10 (m, 5H), 1.76-1.58 (m,3H).

### Example 4.

### Synthesis of 3β-[4-(N-t-Boc-Indol-5-yl)-phenyl]-tropane-2β-carboxylic acid methyl ester (22).

Synthesis of this compound followed generally Scheme II:

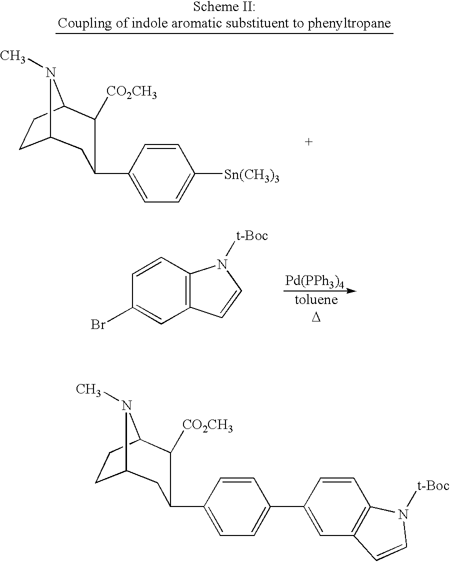

To a solution of 200 mg (0.475 mmol) of 3-(4-trimethylstannanyl-phenyl)-tropane-2β-carboxylic acid methyl ester in 5 mL of dry toluene under a nitrogen atmosphere was added 169 mg (0.57 mmol) of 5-bromo-indole-1-carboxylic acid tert-butyl ester followed by 32 mg tetrakis (triphenylphosphine) palladium. The mixture was heated to reflux for 18 h. The reaction mixture was purified by flash chromatography on silica gel. Elution with 70:30 hexane-ether with 5% additional triethylamine afforded 80 mg (36% yield) of 3β-[4-(N-t-Boc-Indol-5-yl)-phenyl]-tropane-2β-carboxylic acid methyl ester. ¹H NMR (CD₂Cl₂) δ 8.23 (d, 1H J=8 Hz), 7.82 (d, 1H, J=1.6 Hz), 7.68 (d, 1H, J=4 Hz), 7.62-7.59 (m, 3H), 7.38 (d, 2H,J=8.4), 6.67 (d, 1H, J=3.2 Hz), 3.628 (m, 1H), 3.62 (s, 3H), 3.40 (m, 1H), 3.08 (m, 2H), 2.60 (dt, 1H), 2.25 (m, 5H) 1.79-1.67 (m, 12H).

### Example 5.

### Synthesis of 3β-(3′-methoxybiphenyl-4-yl)nortropane-2β-carboxylic acid methyl ester (10)

Synthesis of this compound followed generally Scheme I above where R═H, Z═C, X═OCH₃. To a solution of 120 mg (0.228 mmol) of 3β-(3′-Methoxy-biphenyl-4-yl)nortropane-2β,8-dicarboxylic acid 2-methyl ester 8-(2,2,2-trichloro-ethyl) ester in 3 mL of dry THF under a nitrogen atmosphere was added 3 mL of 1 M aqueous ammonium acetate followed by 152 mg (1.141 mmol) 10% Cd/Pb. The mixture was stirred at room temperature for 2 h and then filtered through a plug of Celite. The solution was basified with ammonium hydroxide and extracted with ether (5×). The combined extract was dried over magnesium sulfate, filtered, and concentrated under reduced pressure. The crude oil was purified by flash chromatography on silica gel. Elution with 90:10 ether-triethylamine afforded 60 mg (75% yield) of 3β-(3′-methoxybiphenyl-4-yl)nortropane-2β-carboxylic acid methyl ester. The product was analyzed as follows: mp 102-4° C. ¹H NMR (CD₂Cl₂) δ 7.56 (d, 2H J=7.5 Hz), 7.35-7.14 (m, 5H), 6.90 (d, 1H, J=7.2 Hz), 3.85 (s, 3H), 3.71 (d, 2H), 3.40 (s, 3H), 3.31 (m, 1H), 2.82 (d, 1H), 2.44 (t, 1H), 2.15-1.88 (m, 2H), 1.85-1.60 (m, 3H). Anal. (C₂₂H₂₅NO₃) C, H, N.

### Example 6.

### Synthesis of 3β-[3′-(tert-Butoxycarbonylaminomethyl)biphenyl-4-yl]-Tropane-2β-carboxylic acid methyl ester (6)

Synthesis of this compound followed generally Scheme I, where R═CH₃, Z═C, X═CH₂NH-t-Boc:

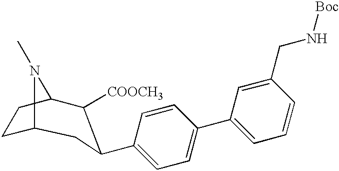

To the solution of 200 mg (0.475 mmol) of 3-(4-trimethylstannanyl-phenyl)-tropane-2β-carboxylic acid methyl ester in 5 mL of dry toluene under a nitrogen atmosphere was added 188 mg (0.57 mmol) of (3-iodobenzyl)-carbamic acid tert-butyl ester followed by 32 mg tetrakis (triphenylphosphine) palladium. The mixture was heated to reflux for 18 h. The reaction mixture was purified by flash chromatography on silica gel. Elution with 60:40 hexane-ether with 5% additional triethylamine afforded 14.3 mg (6% yield) of 3β-[3′-(tert-butoxycarbonyl-aminomethyl)biphenyl-4-yl]-tropane-2β-carboxylic acid methyl ester. The product was analyzed as follows: ¹H NMR (CD₂Cl₂) δ 7.53 (m, 4H), 7.39 (t, 1H, J=7.8 Hz), 7.34 (d, 2H, J=8.34 Hz), 7.25 (d, 1H, J=7.5 Hz), 4.35 (d, 1H), 3.57 (m, 1H), 3.49 (s, 3H), 3.35 (m, 1H), 3.10-2.95 (m, 2H), 2.54 (m, 1H), 2.24-2.10 (m, 5H), 1.80-1.59 (m, 3H), 1.45 (s, 9H).

### Example 7.

### Synthesis of 3β-(4-Thiophen-2-yl-phenyl)tropane-2β-carboxylic acid methyl ester (11)

Synthesis of this compound followed generally Scheme III:

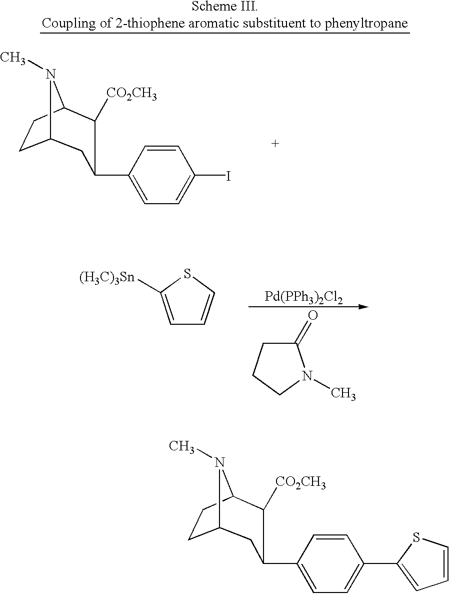

To a solution of 100 mg (0.260 mmol) of 3β-(4-iodophenyl)tropane-2β-carboxylic acid methyl ester in 4 mL of 1-methyl-2-pyrrolidinone under a nitrogen atmosphere was added 145 mg (0.390 mmol) of trimethyl-thiophen-2-yl-stannanefollowed by 18 mg dichloro-bis (triphenyl-phosphine) palladium. The mixture was stirred at room temperature for 72 h. The reaction mixture was purified by flash chromatography on silica gel. Elution with 40:60 hexane-ether with 5% additional triethylamine afforded 44 mg (50% yield) of 3β-(4-thiophen-2-yl-phenyl)tropane-2β-carboxylic acid methyl ester. The product was analyzed as follows: ¹H NMR (CD₂Cl₂) δ 7.53 (d, 2H J=8.1 Hz), 7.31-7.24 (m, 4H), 7.08 (q, 1H), 3.55 (m, 1H), 3.47 (s, 3H), 3.34 (m, 1H), 3.00 (m, 2H), 2.52 (dt, 1H), 2.25-2.07 (m,5H), 1.77-1.60 (m, 3H).

### Example 8

### 3β-(4-Thiophen-3-yl-phenyl)tropane-2β-carboxylic acid methyl ester (12)

Synthesis of this compound followed generally Scheme IV:

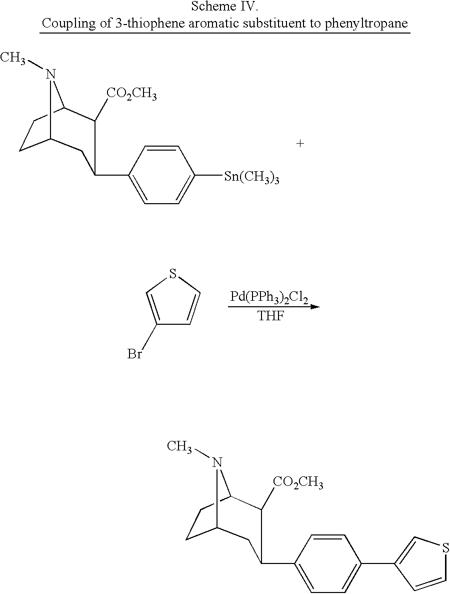

To a solution of 100 mg (0.237 mmol) of 3-(4-trimethylstannyl-phenyl)tropane-2β-carboxylic acid methyl ester in 5 mL of dry tetrahydrofuran under a nitrogen atmosphere was added 46.4 mg (0.284 mmol) of 3-bromo-thiophene followed by 8.3 mg dichloro bis (triphenylphosphine) palladium. The mixture was stirred at room temperature for 72 h. The reaction mixture was purified by flash chromatography on silica gel. Elution with 40:60 hexane-ether with 5% additional triethylamine afforded 12.5 mg (15% yield) of 3β-(4-thiophen-3-yl-phenyl)tropane-2β-carboxylic acid methyl ester. The product was analyzed as follows: ¹H NMR (CD₂Cl₂) δ 7.54 (d, 2H, J=8.4 Hz), 7.45 (t, 1H), 7.40 (m, 2H), 7.30 (d, 2H, J=8.1 Hz), 3.57 (m,1H), 3.47 (s, 3H), 3.35 (m, 1H), 3.00 (m, 2H), 2.54 (dt, 1H), 2.30-2.05 (m,5H), 1.80-1.60 (m, 3H) anal. (C₂₀H₂₃NO₂S) C, H, N.

### Example 9

### 3β-(4-Thiazol-2-yl-phenyl)tropane-2β-carboxylic acid methyl ester (14)

Synthesis of this compound followed generally Scheme V:

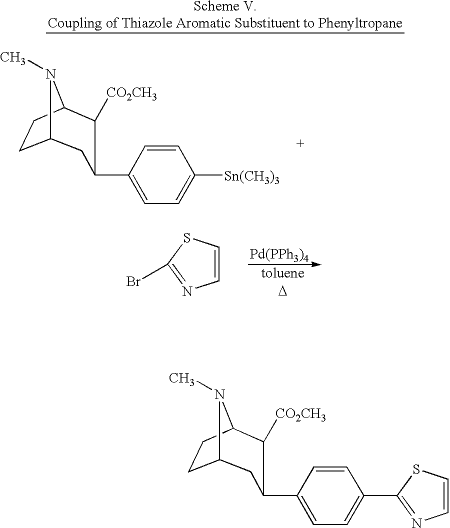

To a solution of 200 mg (0.475 mmol) of 3-(4-trimethylstannanyl-phenyl)-tropane-2β-carboxylic acid methyl ester in 5 mL of dry toluene under a nitrogen atmosphere was added 93 mg (0.57 mmol) of 2-bromo-thiazole followed by 32 mg tetrakis (triphenylphosphine) palladium. The mixture was heated to reflux for 96 h. The reaction mixture was purified by flash chromatography on silica gel. Elution with 60:40 hexane-ether with 5% additional triethylamine afforded 45 mg (28% yield) of 3β-(4-thiazol-2-yl-phenyl)-Tropane-2β-carboxylic acid methyl ester. The product was analyzed as follows: ¹H NMR (CD₂Cl₂) δ 7.91 (d, 2H, J=8 Hz), 7.86 (d, 1H, J=4 Hz), 7.37 (dd, 3H, J=8 Hz, J=3.6 Hz), 3.62 (q, 1H), 3.50 (s, 3H), 3.10-3.00 (m, 2H), 2.57 (dt, 1H), 2.25-2.13 (m, 5H), 1.81-1.63 (m, 3H).

### Example 10

### 3β-(3′-Methoxybiphenyl-4-yl)nortropane-2β,8-dicarboxylic acid 2-methyl ester 8-(2,2,2-trichloro-ethyl) ester

Synthesis of this compound followed generally Scheme VI:

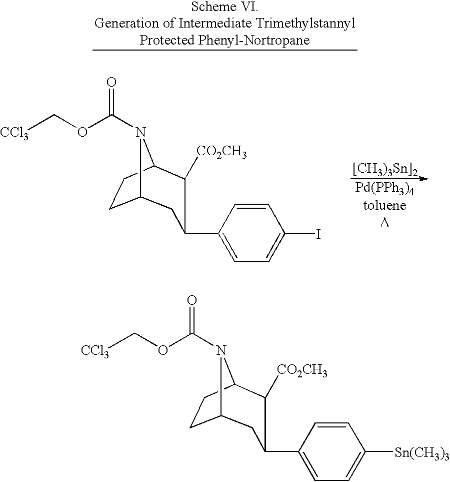

To a solution of 350 mg (0.638 mmol) of 3β-(4-trimethylstannylphenyl)nortropane-2β, 8-dicarboxylic acid 2-methyl ester 8-(2,2,2-trichloroethyl) ester in 7 mL of dry toluene under a nitrogen atmosphere was added 142 mg (0.765 mmol) of 1-bromo-3-methoxy-benzene followed by 22 mg dichloro-bis(triphenylphosphine)palladium. The mixture was heated at reflux for 12 h. The reaction mixture was purified by flash chromatography on silica gel. Elution with 65:35 cyclohexane-ether afforded 150 mg (44% yield) of 3β3-(3′-methoxy-biphenyl-4-yl)nortropane-2β,8-dicarboxylic acid 2-methyl ester 8-(2,2,2-trichloro-ethyl) ester. The product was analyzed as follows: ¹H NMR (CD₂Cl₂) δ 7.55 (d, 2H, J=8.1 Hz), 7.34 (m, 3H), 7.16 (m, 2H), 6.89 (dd, 1H), 4.95-4.40 (m, 4H), 3.85 (s, 3H), 3.50-3.35 (m, 4H), 3.05 (m, 1H), 2.82 (m, 1H), 2.30-1.75 (m, 5H).

### Example 11

### 3β-(4-Trimethylstannylphenyl)nortropane-2β, 8-dicarboxylic acid 2-methyl ester 8-(2,2,2-trichloro-ethyl) ester

Synthesis of this compound followed generally Scheme VI above. To a solution of 500 mg (0.917 mmol) of 3β-(4-iodophenyl)nortropane-2β, 8-dicarboxylic acid 2-methyl ester 8-(2,2,2-trichloroethyl) ester in 10 mL of dry toluene under a nitrogen atmosphere was added 600 mg (1.834 mmol) of hexamethylditin followed by 52 mg tetrakis(triphenylphosphine) palladium. The mixture was heated at reflux for 18 h. The reaction mixture was purified by flash chromatography on silica gel. Elution with hexane with 5% additional triethylamine afforded 392 mg (74% yield) of 3β-(4-trimethylstannanyl-phenyl)nortropane-2β, 8-dicarboxylic acid 2-methyl ester 8-(2,2,2-trichloro-ethyl) ester; mp138-9° C. The product was analyzed as follows: ¹H NMR (CD₂Cl₂) δ 7.43 (d, 2H, J=7.8 Hz), 7.21 (d, 2H, J=7.5 Hz), 4.97-4.38 (m, 4H), 3.42 (s, 3H), 3.30 (m, 1H), 2.96 (m, 1H), 2.76 (m, 1H), 2.27-2.02 (m, 2H), 1.95 (m, 1H), 1.83-1.70 (m,2H), 0.257 (s, 9H). Anal (C₂₁H₂₈Cl₃NO₄Sn) C, H, N

### Example 12

### [4-(5-Methoxy-pyridin-3-yl)-phenyl]nortropane-2β, 8-dicarboxylic acid 2-methyl ester 8-(2,2,2-trichloro-ethyl) ester

Synthesis of this compound followed generally Scheme VII.

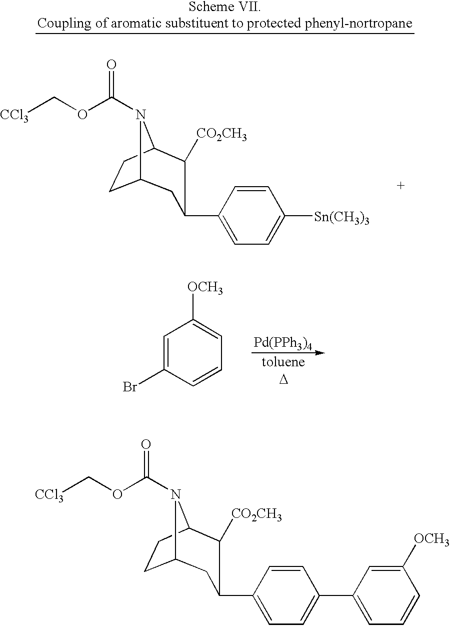

To a solution of 400 mg (0.688 mmol) of 3β-(4-trimethylstannylphenyl)nortropane-2β, 8-dicarboxylic acid 2-methyl ester 8-(2,2,2-trichloroethyl) ester in 5 mL of dry toluene under a nitrogen atmosphere was added 154 mg (0.827 mmol) of 3-bromo-5-methoxy-pyridine followed by 32 mg tetrakis (triphenylphosphine)palladium. The mixture was heated to reflux for 12 h. The reaction mixture was purified by flash chromatography on silica gel. Elution with 60:40 hexane-ether with 5% additional triethylamine afforded 120 mg (33% yield) of 3β-[4-(5-methoxypyridin-3-yl)-phenyl]nortropane-2β, 8-dicarboxylic acid 2-methyl ester 8-(2,2,2-trichloro-ethyl) ester. The product was analyzed as follows: ¹H NMR (CD₂Cl₂) δ 8.46 (d, 1H, J=1.8 Hz), 8.29 (d,1H, J=2.8 Hz), 7.58 (d, 2H, J=6.6 Hz), 7.41 (m, 3H), 4.98-4.48 (m, 4H), 3.92 (s, 3H), 3.42 (m, 4H), 3.06 (m, 1H), 2.86 (m, 1H), 2.39-2.10 (m, 2H), 2.00 (m, 1H), 1.98 (m, 2H).

### Example 13

### 3β-[4-(5-Methoxy-pyridin-3-yl)-phenyl]-nortropane-2β-carboxylic acid methyl ester

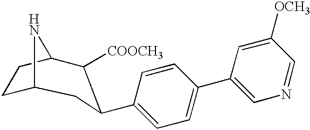

To a solution of 120 mg (0.227 mmol) of 3β-[4-(5-Methoxy-pyridin-3-yl)-phenyl]-nortropane-2β, 8-dicarboxylic acid 2-methyl ester 8-(2,2,2-trichloro-ethyl) ester in 3 ml of dry THF under a nitrogen atmosphere was added 3 ml of 1M aqueous ammounium acetate followed by 152 mg(1.141 mmol) 10% Cd/Pb. The mixture was stirred at room temperature for 2 hrs and then filtered through a plug of Celite. The solution was basified with ammonium hydroxide and extracted with ether, the combined extracts were dried over magnesium sulfate, filtered, and the solvent was removed on a rotary evaporator, and the residue was purified on a silica gel column (5% Et₃N/45%Et₂0/50% hexane) to yield an colorless oil 50 mg (63% yield). The product was analyzed as follows: ¹H NMR (CD₂Cl₂) δ 8.33(d, 1H J=2 Hz), 8.15(d, 1H J=2.8 Hz), 7.44(d, 2H, J=8 Hz), 7.28(q, 1H, J1=2 Hz, J2=2.6 Hz), 7.23(d, 2H, J=8 Hz), 3.80(s, 3H), 3.60(d, 2H), 3.22(s, 3H), 3.19(m, 1H), 2.71(m, 1H), 2.32(dt, 1H), 1.97-1.83(m, 2H), 1.72-1.1.54(m, 3H).

### Example 14

### 3β-[4-(5-amino-thiophen-2-yl)-phenyl]tropane-2β-carboxylic acid methyl ester

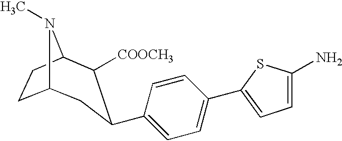

To an ice cooled solution of 77 mg (0.199 mmol) of 3p-[4-(5-nitro-thiophen-2-yl)-phenyl]tropane-2β-carboxylic acid methyl ester in 10 ml of methanol under a nitrogen atmosphere was added 26 mg (0.398 mmol) of dust zinc following 1 ml of 37% hydrochloric acid dropwise. The mixture was stirred at room temperature for 3 h. The solution was basified with ammonium hydroxide and extracted with dichloromethane, the combined extracts were dried over magnesium sulfate, filtered, and the solvent was removed on a rotary evaporator, and the residue was purified on a silica gel column (5% Et₃N/75%Et₂0/20% hexane) to yield an colorless oil 23 mg (27% yield). The product was analyzed as follows: ¹H NMR (CD₂Cl₂) δ 7.32 (d, 2H J=8 Hz, aryl H), 7.20 (d, 2H J=8 Hz, aryl H), 7.00 (d, 1H, J=5.25 aryl H), 6.55 (d, 1H, J=5.25 aryl H), 3.47 (m, 1H), 3.39 (s, 3H), 3.25 (m, 1H), 2.94 (m, 1H), 2.86 (m, 1H), 2.45 (dt, 1H), 2.13-2.00 (m, 5H), 1.67-1.51 (m, 3H).

### Example 15

### 3β-[4-(4-bromo-thiophen-3-yl)-phenyl]tropane-2β-carboxylic acid methyl ester

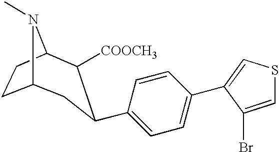

To a solution of 200 mg (0.475 mmol) of β-(4-trimethylstannylphenyl)tropane-2β-carboxylic acid methyl ester in 5 ml of THF under a nitrogen atmosphere was added 574 mg (2.375 mmol) of 3,4-dibromothiophene followed by 64 mg dichlorobis(triphenylphosphine) palladium. The mixture was refluxed for 12 h. The residue was purified on a silica gel column (5% Et₃N/20%Et₂0/75% hexane) to yield an colorless oil 84 mg (42% yield). The product was analyzed as follows: ¹H NMR (CD₂Cl₂) δ 7.48 (d, 2H J=8 Hz, aryl H), 7.44 (d, 1H J=3.6 Hz, aryl H), 7.37 (d, 2H J=8 Hz, aryl H), 7.33 (d, 1H, J=3.6 Hz, aryl H), 3.64 (m, 1H), 3.54 (s, 3H), 3.40 (m, 1H), 3.12-3.02 (m, 2H), 2.63 (td, 1H), 2.31-2.14 (m, 5H), 1.83-1.75 (m, 2H), 1.70-1.64 (m, 1H).

### Example 16

### 3β-[4-(5-bromo-thiophen-2-yl)-phenyl]tropane-2β-carboxylic acid methyl ester

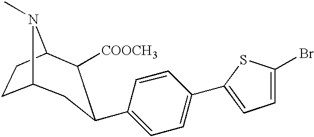

To a solution of 5-bromo-thiophenyl zinc bromide (1 mmol) in 2 ml of THF under a nitrogen atmosphere was added 200 mg (0.519 mmol) of β-CIT followed by 30 mg dichlorobis(triphenylphosphine) palladium. The mixture was stirred at room twmperature for 3 h. The residue was purified on a silica gel column (5% Et₃N/25%Et₂0/70% hexane) to yield an colorless oil 106 mg (49% yield). The product was analyzed as follows: ¹H NMR (CD₂Cl₂) δ 7.48 (d, 2H J=8.2 Hz, aryl H), 7.30 (d, 2H J=8.2 Hz, aryl H), 7.09 (q, 2H, aryl H), 3.61 (m, 1H), 3.50 (s, 3H), 3.37 (m, 1H), 3.05-2.96 (m, 2H), 2.58 (dt, 1H), 2.26-2.12 (m, 5H), 1.79-1.64(m, 3H).

### Example 17

### Binding of Aromatic Substituted Phenyltropane Derivatives to Monoamine Transporters

The compounds of the present invention were analyzed for their ability to bind to the serotonin transporter (SERT), norepinephrine transporter (NET), and dopamine transporter (DAT). For SERT and NET assays, rat cerebral cortex was homogenized in cold SERT assay buffer (50 mM Tris HCl, pH 7.4, 120 mM NaCl, 5 mM KCl) by Polytron (setting 5, 15 sec), centrifuged (10 min, 4° C., 30,000 g), re-suspended in fresh buffer, centrifuged and stored in buffer at −70° C. Before use in the SERT assay, each sample was homogenized by hand to provide the equivalent of 3 mg original tissue per assay tube. For NET assay, the thawed homogenate was suspended in NET assay buffer (SERT buffer containing 300 mM NaCl) to 13.3 mg per tube. For DAT assays, corpus striatum tissue from rat forebrain was rapidly dissected on ice, pooled, weighed, and homogenized at 30 mg/mL in cold DAT buffer (50 mM Tris citrate, pH 7.4, 120 mM NaCl, 4 mM MgCl₂) and stored at −70° C. The pellet was suspended in the assay buffer to provide the equivalent of about 1 mg of wet weight per assay tube.

Test agents were evaluated at 6-12 concentrations, in triplicate, and with independent duplication, using the brain membrane homogenates in the presence of a selective, high-affinity radioligand (DuPont-NEN, Boston Mass.; at concentration=L), with and without a blank agent, as follows: SERT, [³H]paroxetine (20 Ci/mmol; K_{d}=150 pM; L=200 pM) and 2 μM fluoxetine (donated by Lilly Labs, Indianapolis, Ind.) as blank; (Habert, E. et al., Eur. J. Pharmacol. 118: 107-114, 1985); NET, [³H]nisoxetine (50 Ci/mmol; K_{d}=800 pM; L=600 pM) and 2 μM desipramine (Research Biochemicals International, Natick, Mass.) as blank (Tejani-Butt et al., J. Pharmacol. Exp. Ther. 260: 427-436, 1992); DAT, [³H]GBR-12935 (13 Ci/mmol; K_{d}=1.0 nM. L=400 pM) and 1.0 μM GBR-12909 (Research Biochemicals International, Natick, Mass.) as blank (Kula, N. S. et al., Neuropharmacology. 30: 89-92, 1990; Andersen, P. H., J. Neurochem. 48:1887-1896, 1987).

Assay tubes were incubated at room temperature for 120 min (SERT) or on ice for 180 min (NET) or 45 min (DAT). Labeled tissue samples were recovered in a Brandel Cell Harvester on glass fiber filter sheets saturated with 0.3% (v/v) aqueous polyethyleneimine, washed with ice-cold 0.9% NaCl, and counted for tritium in a liquid scintillation counter. Concentration-inhibition curves were computer-fit to determine IC₅₀±SEM and converted to Ki values in nM from the Cheng -Prusoff relationship Ki=IC₅₀/(1+[L]/K_{d}). Selectivity for SERT was calculated as the ratio of Ki for NET or DAT to that for SERT (that is, a larger number is more selective for SERT). The results are shown in Table 6, 7, 8, and 9 for biphenyl derivatives, heterocyclic 5-right derivatives, heterocyclic 6-ring derivatives, and heterocyclic 10-ring derivatives, respectively.

TABLE 6
Biphenyl Derivatives

| | General | | | | | | Selec- |
|---|---|---|---|---|---|---|---|
| Compound | Structure | Position | X | SERT | DAT | NET | tivity |
| 1 | II | — | H | | | | |
| 2 | II | 3″ | OH | 1.39 | 15.2 | 155 | 11.0 |
| 3a | II | 2″ | OCH₃ | 79.5 | 357 | >10,000 | 4.49 |
| 3b | II | 3″ | OCH₃ | 1.89 | 58.7 | >3,000 | 31.1 |
| 3c | II | 4″ | OCH₃ | 32.0 | 13.2 | 352 | 0.41 |
| 4a | II | 2″ | NO₂ | >3,000 | >3,000 | >3,000 | 1 |
| 4b | II | 3″ | NO₂ | 0.80 | 9.54 | 963 | 11.9 |
| 4c | II | 4″ | NO₂ | 29.7 | 5.10 | 370 | 0.17 |
| 5 | II | 3″ | NH₂ | 67.3 | 24.2 | 661 | 0.36 |
| 6a | II | 2″ | COCH₃ | 5,000 | 1,016 | >20,000 | 0.20 |
| 6b | II | 3″ | COCH₃ | 42.0 | 12.4 | >50,000 | 0.30 |
| 6c | II | 4″ | COCH₃ | 179 | 19.1 | 754 | 0.11 |
| 7 | — | 3″ | CH₂NH-tBoc | >10,000 | >10,000 | >30,000 | 1 |
| 8 | — | 3″ | COPh | >3,000 | >3,000 | >3,000 | 1 |
| 9 | — | 3″ | CH(OCH₂) | >10,000 | 363 | >30,000 | 0.03 |
| 10 | — | 3″ | OCH₃ | 2.32 | 710 | 295 | 306 |

TABLE 7
Heterocyclic 5-ring Derivatives

| | General | Pos- | | | | | |
|---|---|---|---|---|---|---|---|
| Compound | Structure | ition | X | SERT | DAT | NET | Selectivity |
| 11 | IIIa | 2″ | S | 0.15 | 52 | 158 | 346 |
| 12 | IIIb | 3″ | S | 0.17 | 12.1 | 189 | 711 |
| 13 | IIIa | 2″ | O | 1.13 | 7.14 | 1,399 | 6.32 |
| 14 | IIIc | — | S, N | 1.67 | 520 | >10,000 | 310 |

TABLE 8
Heterocyclic 6-ring Derivatives

| Comp- | General | | Posi- | | | | | |
|---|---|---|---|---|---|---|---|---|
| ound | Structure | R | tion | X | SERT | DAT | NET | Selectivity |
| 15 | IVa | CH₃ | 2″ | H | 161 | 44.0 | 10,000 | 0.27 |
| 16 | IVb | CH₃ | 3″ | H | 3.54 | 26.5 | 1,407 | 7.3 |
| 17 | IVb | CH₃ | 4″ | OCH₃ | | | | |
| 18 | IVb | H | 3″ | OCH₃ | 3.91 | 587 | 1,594 | 150 |
| 19 | — | CH₃ | — | — | 33.1 | 344 | >10,000 | 10.4 |

TABLE 9
Heterocyclic 10-ring Derivatives

| Compound | Type | SERT | DAT | NET | Selectivity |
|---|---|---|---|---|---|
| 20 | Quinoline | 20.0 | 59.8 | >3,000 | 3.0 |
| 21 | Isoquinoline | 3,000 | >30,000 | >10,000 | 10 |
| 22 | Indole | | | | |

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

What is claimed is:

1. A tropane analog having the structure (I) 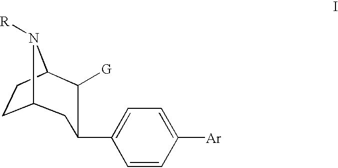
wherein Ar is linked to said structure (I) through the dotted line and is selected from the group consisting of 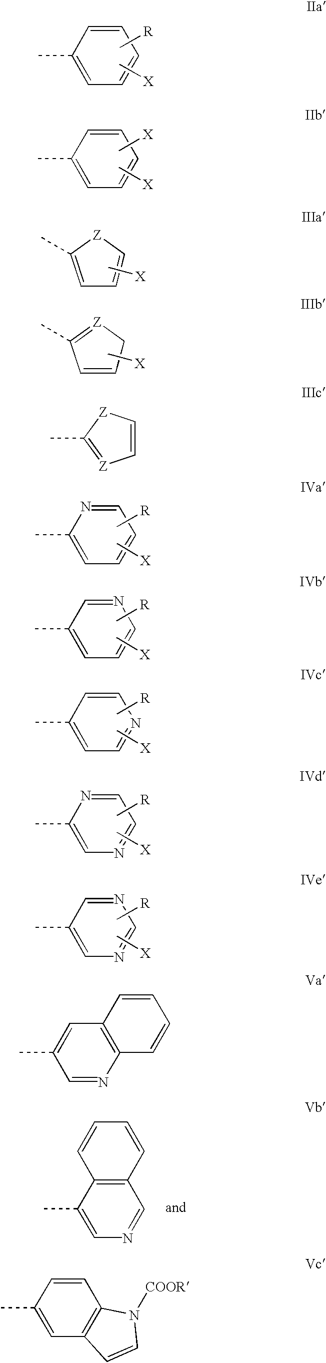
and wherein:
G is CO₂R, COR, CH₂OR, or CH═CHR;
X is H, I, Br, Cl, F, NO₂, COR, or, NH₂;
R and R′ are each individually H or a substituted or unsubstituted lower alkyl group; and
Z═O, S, or N.

2. The tropane analog of claim 1, having the structure 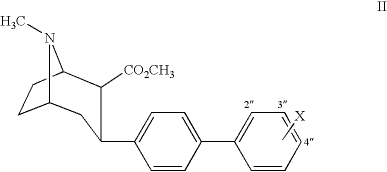
wherein X is selected from the group consisting of H, OH, OCH₃, NO₂, NH₂, COCH₃, CH₂NHtBoc, COC₆H₅, and CH(OCH₂)₂.

3. A tropane analog having the structure of

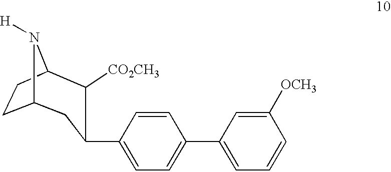

4. The tropane analog of claim 1, having a structure selected from the group consisting of 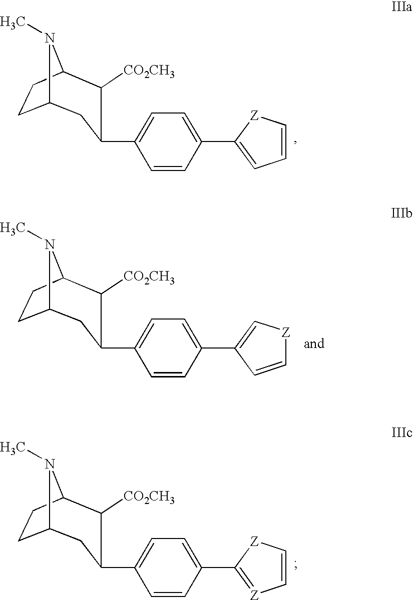
wherein Z is S, O, or N.

5. The tropane analog of claim 1, having a structure selected from the group consisting of 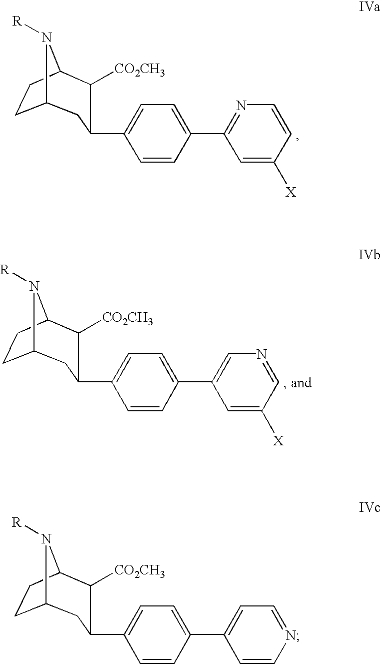
wherein R is H or CH₃, and X is H or OCH₃.

6. The tropane analog of claim 1, having a structure selected from the group consisting of

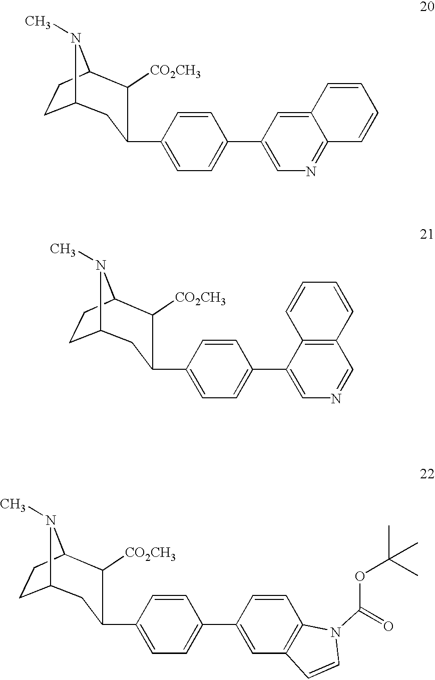

7. The tropane analog of claim 1, wherein the halogen atoms of X are heavy or radioactive isotopes.

8. The tropane analog of claim 7, wherein said heavy or radioactive isotopes are selected from the group consisting of ³H, ¹²³I, ¹²⁵I, ¹³¹I, ¹⁸F, ⁷⁶Br, ¹¹C, ¹⁴C, and combinations thereof.

9. The tropane analog of claim 1, wherein said tropane analog binds to the serotonin transporter, norepinephrine transporter, or dopamine transporter in the brain of a mammal.
